(19) United States
(12) Patent Application Publication
(10) Pub. No.: US 2017/0014028 A1
(43) Pub. Date: Jan. 19, 2017

(54) MEDICAL MONITORING AND TREATMENT DEVICES, SYSTEMS, AND METHODS

(71) Applicant: Gerald E. Clear, JR., Camden, IN (US)

(72) Inventor: Gerald E. Clear, JR., Camden, IN (US)

(21) Appl. No.: 15/209,073

(22) Filed: Jul. 13, 2016

Related U.S. Application Data
(60) Provisional application No. 62/191,771, filed on Jul. 13, 2015.

(51) Int. Cl. A61B 5/00; G06F 19/00; A61M 5/172; A61B 5/11; A61B 5/145; A61B 5/117

(52) U.S. Cl. CPC A61B 5/0022; A61B 5/14542; A61B 5/14532; A61B 5/117; A61B 5/4839; A61B 5/746; A61B 5/7405; A61B 5/7455; A61M 5/1723; A61B 5/1112; G06F 19/3418; A61M 2205/3553; A61M 2205/3592

(57) ABSTRACT

Medical monitoring and treatment devices, systems, and methods. In one system embodiment, the system comprises a medical device configured to obtain a bodily measurement from a patient; an alert device comprising a receiver configured to receive a signal from the medical device, the signal indicative of the bodily measurement; and at least one user device in communication with the alert device; the alert device configured so that when the bodily measurement falls outside of a predetermined bodily measurement range, the alert device is operable to send an alert signal to the at least one user device, the alert signal indicative of the bodily measurement falling outside of the predetermined bodily measurement range.

### PRIORITY

The present application is related to, and claims the priority benefit of, U.S. Provisional Patent Application Ser. No. 62/191,771, filed Jul. 13, 2015, the contents of which are hereby incorporated into the present application in their entirety.

### BACKGROUND

Millions of Americans and other people around the world suffer from conditions related to blood sugar levels that are either too low or too high, and/or suffer from conditions related to blood oxygen levels being too low, such as various chronic or acute conditions. Should said conditions cause the person to become dizzy, lose consciousness, etc., the person could be at risk for worsening the condition, or even death.

In view of the same, systems and methods to use the same to help monitor patients and alert caregivers, family members, and the like, would be well received in the marketplace.

### BRIEF SUMMARY

In at least one embodiment of a system of the present disclosure, the system comprises a medical device configured to obtain a bodily measurement from a patient; an alert device comprising a receiver configured to receive a signal from the medical device, the signal indicative of the bodily measurement; and at least one user device in communication with the alert device; the alert device configured so that when the bodily measurement falls outside of a predetermined bodily measurement range, the alert device is operable to send an alert signal to the at least one user device, the alert signal indicative of the bodily measurement falling outside of the predetermined bodily measurement range.

In at least one embodiment of a system of the present disclosure, the medical device is selected from the group consisting of a glucose monitoring system, a glucometer, an insulin pump, and an oxygen monitor.

In at least one embodiment of a system of the present disclosure, the medical device further comprises a location mechanism, the location mechanism configured to identify a location of the medical device.

In at least one embodiment of a system of the present disclosure, the medical device comprises a glucose monitoring system, wherein the bodily measurement is a blood glucose measurement, and wherein the predetermined bodily measurement range comprises a range of acceptable blood glucose measurements.

In at least one embodiment of a system of the present disclosure, the medical device comprises an oxygen monitor, wherein the bodily measurement is a blood oxygen measurement, and wherein the predetermined bodily measurement range comprises a range of acceptable blood oxygen measurements.

In at least one embodiment of a system of the present disclosure, the alert device is operable to send an alert signal to the at least one user device through a network.

In at least one embodiment of a system of the present disclosure, the alert device further comprises a transmitter, wherein the transmitter of the alert device is operable to send an alert signal to the at least one user device.

In at least one embodiment of a system of the present disclosure, the alert device comprises a storage medium having software stored thereon, a processor in communication with the storage medium and operable to perform instructions within the software, the software configured to compare the bodily measurement to the predetermined bodily measurement range to determine whether or not the bodily measurement falls within or outside of the predetermined bodily measurement range.

In at least one embodiment of a system of the present disclosure, the at least one user device comprises a smartphone or smartwatch and is configured to display information indicative of the alert signal.

In at least one embodiment of a system of the present disclosure, the alert signal is further indicative of an identification of the patient.

In at least one embodiment of a system of the present disclosure, the alert signal is further indicative of the bodily measurement falling outside of the predetermined bodily measurement range.

In at least one embodiment of a method of the present disclosure, the method comprises the steps of operating a medical device configured to obtain a bodily measurement from a patient to obtain the bodily measurement; operating an alert device comprising a receiver configured to receive a signal from the medical device, the signal indicative of the bodily measurement; comparing the bodily measurement to a predetermined bodily measurement range using the alert device to determine whether or not the bodily measurement falls within or outside of the predetermined bodily measurement range; and operating the user device to send an alert signal to at least one user device in communication with the alert device, the alert signal indicative of the bodily measurement falling outside of the predetermined bodily measurement range.

In at least one embodiment of a method of the present disclosure, the method further comprises the steps of operating a location device in communication with the medical device to determine a location of the medical device, and further operating the alert device to send a location signal to the at least one user device, the location signal indicative of the location of the medical device.

In at least one embodiment of a method of the present disclosure, the medical device comprises a glucose monitoring system or a glucometer, wherein the bodily measurement is a blood glucose measurement, and wherein the predetermined bodily measurement range comprises a range of acceptable blood glucose measurements.

In at least one embodiment of a method of the present disclosure, the medical device comprises an oxygen monitor, wherein the bodily measurement is a blood oxygen measurement, and wherein the predetermined bodily measurement range comprises a range of acceptable blood oxygen measurements.

In at least one embodiment of a system of the present disclosure, the system comprises a medical device configured to obtain a bodily measurement from a patient, the medical device selected from the group consisting of a glucose monitoring system, a glucometer, an insulin pump, and an oxygen monitor; a location mechanism in communication with the medical device, the location mechanism configured to identify a location of the medical device; an alert device comprising a receiver configured to receive a signal from the medical device and a transmitter configured to transmit an alert signal, the signal indicative of the bodily measurement selected from the group consisting of a blood glucose level and a blood oxygen level; and at least one user device in communication with the alert device, the at least one user device configured to receive the alert signal from the alert device; the alert device configured so that when the bodily measurement falls outside of a predetermined bodily measurement range, the alert device is operable to send the alert signal to the at least one user device, the alert signal indicative of the bodily measurement falling outside of the predetermined bodily measurement range and further indicative of the location of the medical device.

In at least one embodiment of a system of the present disclosure, the alert device is operable to send an alert signal to the at least one user device through a network.

In at least one embodiment of a system of the present disclosure, the alert signal is further indicative of an identification of the patient and a location of the patient.

In at least one embodiment of a system of the present disclosure, the at least one user device is configured to display information indicative of the alert signal.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a component diagram of components of a system, according to an exemplary embodiment of the present disclosure;

FIG. 2 shows a component diagram of portions of an alert device, according to an exemplary embodiment of the present disclosure;

FIG. 3 shows a component diagram of portions of a user device, according to an exemplary embodiment of the present disclosure; and

FIG. 4 shows a component diagram of components of a system configured to communicate over a network, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure of various devices, systems, and methods relating to monitoring and potentially treating people having a condition relating to blood sugar levels (such as diabetes) and/or other conditions, such as chronic obstructive pulmonary disease (COPD).

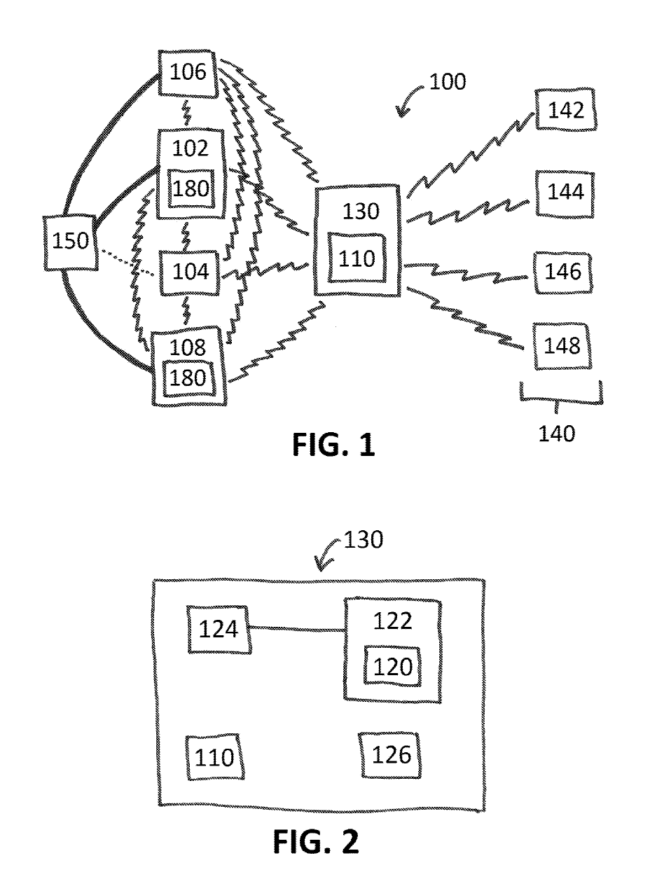

An exemplary embodiment of a system of the present disclosure is described as follows. In at least one embodiment, and as shown in FIG. 1, system 100 comprises a glucose (or blood sugar) monitoring system 102 (configured to be coupled to a patient 150) and/or a glucometer 104 (configured to test blood glucose (sugar) levels and not be coupled to patient 150), whereby at least one of glucose monitoring system 102 and/or glucometer 104 is in communication with a receiver 110 (which may be stand-alone or part of an alert device 130, described in further detail herein). Receiver 110, in various embodiments, may be configured to obtain data from glucose monitoring system 102 and/or glucometer 104, and/or may be further configured to provide instructions to glucose monitoring system 102 and/or glucometer 104.

System 100, in various embodiments, would be operable in view of specific instructions, such as via software 120 stored within a storage medium 122 and accessible using a processor 124 coupled thereto, whereby processor 124 directs performance of instructions/steps within software 120. Software 120, for example, may then include pre-set limits for high blood sugar and/or low blood sugar, so that if receiver 110 is aware that the person using system 100 (also referred to as patient 150) has blood sugar at or higher than the high blood sugar limit or at or lower than the low blood sugar limit, system 100 would operate to perform some sort of task, as described further herein. The high blood sugar and/or the low blood sugar limits can be set based on a particular patient 150, be pre-set in general, etc., and/or can be changed over time as may be desired. For example, if the person using system 100 (patient 150) becomes somewhat disoriented with a blood sugar reading of 75, then, for example, the low blood sugar limit could be set at 85, so to address the needs of patient 150 prior to patient 150 becoming disoriented, for example.

FIG. 1 shows components of an exemplary system 100 of the present disclosure. As shown therein, system 100 comprises an alert device 130, referenced in further detail in FIG. 2, that is in communication with one or both of a glucose monitoring system 102 and/or a glucometer 104. Alert device 130 can communicate with one or more user devices 140, such as smartphones/smartwatches 142, tablets 144, traditional cellular or landline telephones 146, computers 148, etc., which can be used by one or more family members, caregivers, medical personnel, emergency personnel, etc. The jagged lines in FIG. 1 represent signals/alerts, the solid lines represent direct connections, and the dotted line represents blood sugar testing of the patient 150's blood using glucometer 104.

FIG. 2 shows components of an exemplary alert device 130 of the present disclosure, having a processor 124 coupled to a storage medium 122 with software 120 stored thereon. Alert device 130 comprises receiver 110, as referenced herein, which can receive data/information from one or more of glucose monitoring system 102, glucometer 104, and/or one or more user devices 140. As referenced herein, receiver 110 may also be configured to send out (transmit) the signals to user devices 140 and/or other components of system 100 as referenced herein, but in embodiments of system whereby receiver 110 is not configured to send signals, a transmitter 126 may be used to send out said signals.

If a patient 150 is using a continuous glucose monitoring system 102, receiver 110 could, for example, receive the glucose reading every five minutes (or at other time intervals or time frames as may be desired), and once the low blood sugar limit is met, system 100 would automatically send out one or more signals to one or more user devices 140, such as dialing a family member's smartphone/smartwatch 142, a caregiver's phone 146, a medical personnel's smartphone/smartwatch 142, etc., sending a text message, sending an alert to an application on a phone, etc., so to alert one or more people that the patient 150's blood sugar has met or surpassed the low blood sugar limit, or that patient 150 is generally in danger for having low blood sugar. Such a signal could cause, for example, smartphone/smartwatch 142 to ring, vibrate, etc., so to advise the user/wearer of smartphone/smartwatch 142 that patient 150 has low blood sugar and may require attention. Should a person be wearing a watch-like device (an exemplary smartphone/smartwatch 142), a vibration could, for example, wake the person when if asleep, such as if/when the person would otherwise sleep through an auditory signal. This would alert the family member, caregiver, medical personnel, etc., to attempt to contact patient 150 with the low or potentially low blood sugar so to try to help patient 150 correct the problem. Receiver 110 would continue to monitor patient 150's blood sugar readings, and if the readings have not been corrected, or if the readings continue to go lower, system 100 would again send out one or more signals to attempt to alert someone that patient 150 using system 100 may be in need of help. System 100 could also be programmed so that should patient 150's blood sugar not return to a normal or otherwise acceptable range, emergency personnel (such as 911) may be contacted in addition to family, caregivers, medical personnel, etc., as may be needed/desired. The same applies when a patient 150, for example, has met or exceed his or her high blood sugar limits. Signals/alerts can be sent and/or repeated at pre-set times. The vibration or auditory signal could persist until the low sugar concern or high sugar concern is rectified, such as by increasing blood sugar levels to an acceptable level.

If patient 150 is not using a glucose monitoring system 102 and is only using a glucometer 104, receiver 110 would use the readings/signals sent by or received from glucometer 104 and send out one or more signals to family members, caregivers, medical personnel, emergency personnel, etc., as may be desired should patient 150's blood sugar levels be at or below the low blood sugar limit or at or above the high blood sugar limit, as may be applicable. The family members, caregivers, medical personnel, emergency personnel, etc., can then attempt to contact patient 150 and help them through the low or high blood sugar level alert/situation.

An application (“app”) (exemplary software 120 of the present disclosure) on a smartphone/smartwatch 142, tablet 144, computer 148, etc., could be used by one or more family members, caregivers, medical personnel, etc., so that a generally continuous monitoring of patient 150 can be made, or so that, at a minimum, the family members, caregivers, medical personnel, etc., using the app would be alerted should patient 150 approach, meet, or exceed the pre-set low blood sugar limit or the high blood sugar limit. Such an app could provide information regarding the identity of patient 150, blood sugar levels, and the like. If only glucose monitoring systems 102 were used, periodic readings, such as hourly, daily, or more or less frequent readings, could be obtained and transmitted to the one or more family members, caregivers, medical personnel, etc., via text message, such as if only a glucometer 104 were used.

There are several advantages to such exemplary systems 100. Patient 150, as well as the various family members, caregivers, medical personnel, etc., who care about the well-being of patient 150, would have peace of mind with respect to the general health and safety of patient 150. Serious injury, and even death, can be prevented, or the risks of the same generally minimized, as the various family members, caregivers, medical personnel, emergency personnel, etc., can attempt to locate, contact, and/or generally assist patient 150 should patient 150 require such assistance. For example, and should patient 150's blood sugar levels get too low, to the point where patient 150 becomes disoriented or even unconscious, for example, various family members, caregivers, medical personnel, emergency personnel, etc., will be alerted and can take steps to locate and help patient 150. The same would go for high blood sugar levels, which can cause ketoacidosis, coma, and even death, which can be prevented, or the risks of the same generally minimized, when patient 150 uses system 100 as referenced herein.

Receiver 110, in various embodiments, would be programmable and potentially include some sort of input device, or configured for operation in connection with an input device (such as a wired or wireless keyboard or other input device known in the art), whereby the program (software 120) can be configured to set one or both of the low blood sugar limit and/or the high blood sugar limit. Software 120 can also be programmed to direct a signal to specific user devices 140 in a particular order, such as first sending a signal to a family member user device 140 prior to sending a signal to a medical personnel user device 140, for example.

Said systems 100, or portions thereof, may be consider medical devices/equipment, and therefore potentially covered my private or public (governmental) medical insurance and/or other programs.

Other health conditions (other than blood sugar-related conditions, such as diabetes) could benefit from using various system 100 embodiments of the present disclosure. For example, patients with COPD using at-home oxygen monitors could use portions of system 100, whereby alerts/signals could be sent by portions of system 100 to one or more user devices 140 should the patient 150's oxygen levels become too low. In such an event, an oxygen monitor 106 (connected to patient 150) would be used instead of (or along with) glucose monitoring system 104, so that data relating to the patient 150's oxygen levels can be received by receiver 110.

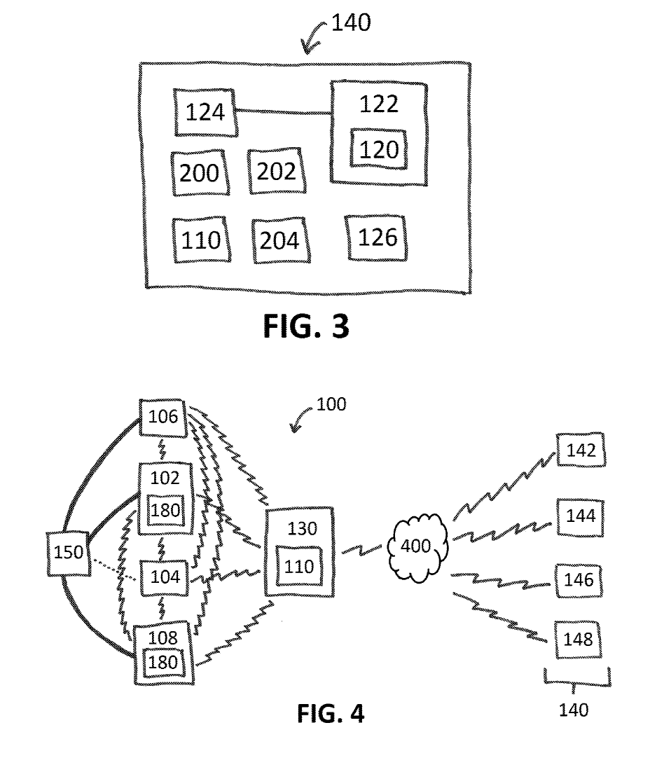

FIG. 3 shows components of an exemplary user device 140 of the present disclosure. As shown therein, user device 140 can also have a processor 124 coupled to a storage medium 122 with software 120 stored thereon. User device 140 can also comprise a receiver 110, as referenced herein, which can receive data/information from one or more of glucose monitoring system 102, glucometer 104, alert device 130, and/or one or more other user devices 140. As referenced herein, receiver 110 may also be configured to send out (transmit) the signals to alert device 130 and/or other user devices 140 and/or other components of system 100 as referenced herein, but in embodiments of system whereby receiver 110 is not configured to send signals, a transmitter 126 may be used to send out said signals. User devices 140 may also comprise one or more of a display 200 configured to display information relating to signals (data/information) from one or more of glucose monitoring system 102, glucometer 104, alert device 130, and/or one or more other user devices 140, a user input mechanism 202, such as a keyboard, touchpad, button, etc., to input information to be sent to one or more system 100 components or to a user device 140 in communication therewith, and a battery 204, such as a rechargeable battery, configured to provide power to various components of user device 140.

Alert devices 130, or components thereof, can be configured for home or use while traveling. As generally referenced herein, alert devices 130 of the present disclosure can communicate with one or more of a glucose monitoring system 102, a glucometer 104, an oxygen sensor 106, and/or an insulin pump 108, and provide data from said items to various user devices 140 of the present disclosure. For example, alert device 130 can provide blood sugar information, oxygen level information, insulin pump information, etc., to one or more devices 140 of the present disclosure, either directly, as shown in FIG. 1, or over a network 400, such as shown in FIG. 4, whereby signals (data/information) from alert device 130 (or directly from one or more of glucose monitoring system 102, glucometer 104, oxygen monitor 106, insulin pump 108, and/or location mechanism 180) can be transmitted to one or more user devices 140 over network 140, such as an intranet, the Internet, and the like.

An insulin pump 108 (configured to administer insulin to a patient 150), as shown in FIG. 1, can also be part of system 100, whereby insulin pump 108 is connected to patient 150 and in communication with alert device 130. Glucose measuring system 102, glucometer 104, oxygen monitor 106, and/or insulin pump 108 can be in communication with one another as well as may be desired. Should alert device 130 identify that patient 150 requires insulin, alert device 130 can send a signal to insulin pump 108 to instruct insulin pump 108 to administer insulin. Should alert device 130 identify that patient 150 is receiving too much insulin, alert device 130 can send a signal to insulin pump 108 to stop insulin pump 108 from administering insulin. Insulin pump 108 could be instructed to stop automatically administering insulin, as noted above, and may also be instructed to prevent the manual administration of insulin by patient 150 as well. This stoppage/prevention may be withdrawn once the patient 150's blood sugar levels return to an acceptable level, for example.

In various embodiments, and as shown in FIG. 1, portions of system 100, such as glucose monitoring system 102, insulin pump 108, etc., may comprise an optional location mechanism 180, so that a particular location of said portion of system 100 can be made. For example, location mechanism 180 may comprise a global positioning system (GPS), a non-GPS locator, etc., whereby one or more portions of system 100, such as, for example, portions of alert device 130 and/or one or more user devices 140 can identify and/or provide the location of glucose monitoring system 102, insulin pump 108, etc., coupled to patient 150. Being able to locate patient 150, for example, can be critical in situations where patient 150 is not in a condition to be able to tell anyone where he/she may be, such as in the event of becoming disoriented or unconscious due to high or low blood sugar or oxygen levels, for example. Receiver 110, for example, may be the part of alert device 130 that is configured to obtain location data, and transmitter 126, for example, may be the part of alert device 130 that is configured to transmit location data to one or more user devices 140. One or more user devices 140 may also be configured to obtain location data directly from the part of system 100 having location mechanism 180.

In various embodiments, portions of system 100, such as alert device 130 or portions thereof, may be password protected or otherwise configured so that persons other than patient 150, such as, for example, or more family members, caregivers, medical personnel, etc., can use portions of system 100 without interference by patient 150. This would prevent patient 150 from in advertently configuring parts of system 100 to the patient 150's detriment, and would also permit or more family members, caregivers, medical personnel, etc., to control the health and well-being of patient 150, such as an elderly or mentally or physically compromised patient 150.

While various embodiments of medical monitoring and treatment devices, systems, and methods have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A system, comprising:
a medical device configured to obtain a bodily measurement from a patient;
an alert device comprising a receiver configured to receive a signal from the medical device, the signal indicative of the bodily measurement; and
at least one user device in communication with the alert device;
the alert device configured so that when the bodily measurement falls outside of a predetermined bodily measurement range, the alert device is operable to send an alert signal to the at least one user device, the alert signal indicative of the bodily measurement falling outside of the predetermined bodily measurement range.

2. The system of claim 1, wherein the medical device is selected from the group consisting of a glucose monitoring system, a glucometer, an insulin pump, and an oxygen monitor.

3. The system of claim 1, wherein the medical device further comprises a location mechanism, the location mechanism configured to identify a location of the medical device.

4. The system of claim 1, wherein the medical device comprises a glucose monitoring system, wherein the bodily measurement is a blood glucose measurement, and wherein the predetermined bodily measurement range comprises a range of acceptable blood glucose measurements.

5. The system of claim 1, wherein the medical device comprises an oxygen monitor, wherein the bodily measurement is a blood oxygen measurement, and wherein the predetermined bodily measurement range comprises a range of acceptable blood oxygen measurements.

6. The system of claim 1, wherein the alert device is operable to send an alert signal to the at least one user device through a network.

7. The system of claim 1, wherein the alert device further comprises a transmitter, wherein the transmitter of the alert device is operable to send an alert signal to the at least one user device.

8. The system of claim 1, wherein the alert device comprises a storage medium having software stored thereon, a processor in communication with the storage medium and operable to perform instructions within the software, the software configured to compare the bodily measurement to the predetermined bodily measurement range to determine whether or not the bodily measurement falls within or outside of the predetermined bodily measurement range.

9. The system of claim 1, wherein the at least one user device comprises a smartphone or smartwatch and is configured to display information indicative of the alert signal.

10. The system of claim 1, wherein the alert signal is further indicative of an identification of the patient.

11. The system of claim 3, wherein the alert signal is further indicative of a location of the patient.

12. The system of claim 1, wherein the alert signal is further indicative of the bodily measurement falling outside of the predetermined bodily measurement range.

13. A method, comprising the steps of:
operating a medical device configured to obtain a bodily measurement from a patient to obtain the bodily measurement;
operating an alert device comprising a receiver configured to receive a signal from the medical device, the signal indicative of the bodily measurement;
comparing the bodily measurement to a predetermined bodily measurement range using the alert device to determine whether or not the bodily measurement falls within or outside of the predetermined bodily measurement range; and
operating the user device to send an alert signal to at least one user device in communication with the alert device, the alert signal indicative of the bodily measurement falling outside of the predetermined bodily measurement range.

14. The method of claim 13, further comprising the steps of:
operating a location device in communication with the medical device to determine a location of the medical device; and
further operating the alert device to send a location signal to the at least one user device, the location signal indicative of the location of the medical device.

15. The method of claim 13, wherein the medical device comprises a glucose monitoring system or a glucometer, wherein the bodily measurement is a blood glucose measurement, and wherein the predetermined bodily measurement range comprises a range of acceptable blood glucose measurements.

16. The method of claim 13, wherein the medical device comprises an oxygen monitor, wherein the bodily measurement is a blood oxygen measurement, and wherein the predetermined bodily measurement range comprises a range of acceptable blood oxygen measurements.

17. A system, comprising:
a medical device configured to obtain a bodily measurement from a patient, the medical device selected from the group consisting of a glucose monitoring system, a glucometer, an insulin pump, and an oxygen monitor;
a location mechanism in communication with the medical device, the location mechanism configured to identify a location of the medical device;
an alert device comprising a receiver configured to receive a signal from the medical device and a transmitter configured to transmit an alert signal, the signal indicative of the bodily measurement selected from the group consisting of a blood glucose level and a blood oxygen level; and
at least one user device in communication with the alert device, the at least one user device configured to receive the alert signal from the alert device;
the alert device configured so that when the bodily measurement falls outside of a predetermined bodily measurement range, the alert device is operable to send the alert signal to
the at least one user device, the alert signal indicative of the bodily measurement falling outside of the predetermined bodily measurement range and further indicative of the location of the medical device.

18. The system of claim 17, wherein the alert device is operable to send an alert signal to the at least one user device through a network.

19. The system of claim 17, wherein the alert signal is further indicative of an identification of the patient and a location of the patient.

20. The system of claim 17, wherein the at least one user device is configured to display information indicative of the alert signal.
